SEQUENCE LISTING (5 sequences)

SEQ ID NO: 1 - protein, 15 aa, synthetic construct
KKWRKVLKLIKRLVG

SEQ ID NO: 2 - protein, 14 aa, synthetic construct
KKWRKVLKFIKRLV

SEQ ID NO: 3 - protein, 13 aa, synthetic construct
KKWRKVLKFIKRL

SEQ ID NO: 4 - protein, 15 aa, synthetic construct
KKWRKVGKLIKRLVG

SEQ ID NO: 5 - protein, 14 aa, synthetic construct
KKWRKVLDLIKRLV

(19) United States
(12) Patent Application Publication
(10) Pub. No.: US 2026/0001913 A1
(43) Pub. Date: Jan. 1, 2026

(54) PEPTIDE EXHIBITING ANTIBACTERIAL AND ANTI-INFLAMMATORY ACTIVITIES AND METHOD FOR TREATING MEDICAL CONDITION WITH THE SAME

(71) Applicant: Metanovas Biotech Inc., San Francisco, CA (US)

(72) Inventors: Lun YU, San Jose, CA (US); Meijie WANG, Santa Clara, CA (US); Heng LUO, Brooklyn, NY (US)

(21) Appl. No.: 19/306,992

(22) Filed: Aug. 21, 2025

Related U.S. Application Data
(63) Continuation-in-part of application No. 19/059,206, filed on Feb. 20, 2025.
(60) Provisional application No. 63/555,503, filed on Feb. 20, 2024.

(51) Int. Cl. C07K 7/08; A61K 38/00

(52) U.S. Cl. CPC C07K 7/08; A61K 38/00

(57) ABSTRACT

A peptide exhibiting both antibacterial and anti-inflammatory activities or a derivative thereof, including an amino acid sequence of SEQ ID NO: 1 or a variant thereof. The peptides synthesized in this disclosure exhibit antibacterial and anti-inflammatory effects, inhibiting one or more of the following microbes: Escherichia coli, Staphylococcus aureus, Propionibacterium acnes, Candida albicans, as well as the expression of the inflammatory factors IL-1β, IL-6, CCL-2, and COX-2.

### CROSS-REFERENCE TO RELATED APPLICATIONS

This application is a continuation-in-part of U.S. application Ser. No. 19/059,206, filed on Feb. 20, 2025, now pending, which is a continuation-in-part of U.S. Ser. No. 63/555,503 filed on Feb. 20, 2024. The contents of all of these specifications are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### INCORPORATION-BY-REFERENCE OF SEQUENCE LISTING

This application comprises a sequence listing, which has been submitted electronically in XML file and is incorporated herein by reference in its entirety. The XML file, created on Jun. 3, 2025, is named SZWK-00102-UUS.xml, and is 5,212 bytes in size.

### BACKGROUND

The present disclosure relates to the field of biomedical peptides, and more specifically, to a peptide exhibiting antibacterial and anti-inflammatory activities, and a method for treating medical condition with the same.

Antimicrobial peptides (AMP) function as a natural host defense mechanism against exogenous pathogenic microorganisms. They disrupt bacterial plasma membrane structure, resulting in cell death. Inflammation is a defensive response of body tissues to injurious stimuli, including infection and tissue damage. Chronic inflammation is a key pathological basis for numerous diseases, such as arthritis, inflammatory bowel disease, and skin diseases.

Conventional antimicrobial peptides exhibiting both antibacterial and anti-inflammatory activities are biocompatible and can inhibit the growth of periodontitis-causing bacteria such as Porphyromonas gingivalis and Aggregatibacter actinomycetemcomitans, and inhibit bacterial biofilm formation. They are highly biocompatible and safe. However, these peptides are ineffective against bacteria such as Propionibacterium acnes, Staphylococcus aureus, Escherichia coli, and Candida albicans.

### SUMMARY

To solve the aforesaid problems, the disclosure provides a peptide or a derivative thereof, comprising an amino acid sequence of SEQ ID NO: 1, or a variant thereof.

In a class of this embodiment, the variant of SEQ ID NO: 1 has ≥88% sequence identity with SEQ ID NO: 1.

In a class of this embodiment, the amino acid sequence of SEQ ID NO: 1 comprises an N-terminus; a hydrogen atom of the N-terminus is substituted by CH₃CH(OH)CO— or R₁—CO—, and R₁ is selected from a group consisting of hydrogen, hydroxyl, amino, an alkyl group, and an alkenyl group.

In a class of this embodiment, the amino acid sequence of SEQ ID NO: 1 comprises a C-terminus; a hydrogen atom of the C-terminus is substituted by —NR₂R₃ or —OR₂, and R₂ and R₃ are independently selected from a group consisting of hydrogen, hydroxyl, amino, an alkyl group, and an alkenyl group.

In a class of this embodiment, the alkyl group is selected from a group consisting of methyl, ethyl, isopropyl, isobutyl, tert-butyl, pentyl, hexyl, heptyl, octyl, decyl, 2-ethylhexyl, 2-methylbutyl, and 5-methylhexyl.

In a class of this embodiment, the alkenyl group is selected from a group consisting of vinyl, linoleyl, and oleyl.

In another aspect, the disclosure provides a pharmaceutical composition, comprising: a medication container and a therapeutically effective amount of the peptide or a derivative thereof.

In a class of this embodiment, the peptide or a derivative thereof is in solid powder form in an amount of 200-2,500 mg per dose.

In a class of this embodiment, the pharmaceutical composition further comprises an aqueous solution contained in the medication container; the peptide or a derivative thereof is dissolved in the aqueous solution at a concentration of 3-30 mg/mL.

The disclosure further provides a method for treating a medical condition in a patient, comprising administering to the patient a therapeutically effective amount of the peptide or a derivative thereof.

In a class of this embodiment, an amount of the peptide or a derivative thereof is administered as a single dose is 200-2,500 mg per dose or 2-60 mg/kg of body weight.

In a class of this embodiment, the medical condition is acne and the peptide or a derivative thereof is administered topically to a treatment area on the patient's skin.

Further provided is a method for preparing a cosmetically or pharmaceutically acceptable salt comprising applying the peptide or a derivative thereof.

In a class of this embodiment, the salt is formed by reacting the peptide or a derivative thereof with an organic base, and the organic base is ethylamine, diethylamine, arginine, lysine, histidine or piperazine.

In a class of this embodiment, the salt is formed by reacting the peptide or a derivative thereof with an inorganic acid or an organic acid; the organic acid is acetic acid, citric acid, malonic acid, maleic acid, tartaric acid, fumaric acid, benzoic acid, succinic acid, oxalic acid, or gluconic acid; and the inorganic acid is hydrochloric acid, sulphuric acid, boric acid or carbonic acid.

The peptides synthesized in the disclosure have antibacterial and anti-inflammatory effects, inhibiting one or more of Escherichia coli, Staphylococcus aureus, Propionibacterium acnes, Candida albicans, as well as inhibiting the expression of IL-6, IL-1β, CCL-2, IL-1 factor and nitric oxide (NO). The minimum inhibitory concentration of the peptides is less than the hemolytic concentration thereof and less than the maximum cytotoxicity-free concentration thereof, which makes it commercially available.

The synthesized peptides with antibacterial and anti-inflammatory effects of the disclosure have a minimum inhibitory concentration range of 12.5 μg/mL against Escherichia coli, 8 μg/mL against Staphylococcus aureus, 2 μg/mL against Propionibacterium acnes, and 50 g/mL against Candida albicans.

### BRIEF DESCRIPTION OF THE DRAWINGS

FIG. 1 shows the inhibition of AMP12 on Propionibacterium acnes according to one embodiment of the disclosure;

FIG. 2A shows the inhibition of AMP12 on Escherichia coli; FIG. 2B shows the inhibition of AMP12 on Staphylococcus aureus; FIG. 2C shows the inhibition of AMP12 variants on Escherichia coli; FIG. 2D shows the inhibition of AMP12 variants on Staphylococcus aureus;

FIG. 3 shows the inhibition of AMP12 on Candida albicans according to one embodiment of the disclosure;

FIG. 4 shows the inhibition of AMP12 on Lactobacillus acidophilus according to one embodiment of the disclosure;

FIG. 5A shows the inhibition of AMP12 on NO expression; FIG. 5B shows the inhibition of AMP12 variants on NO expression; FIG. 5C shows the inhibition of AMP12 on IL-1β expression; FIG. 5D shows the inhibition of AMP12 variants on IL-1β expression; FIG. 5E shows the inhibition of AMP12 variants on IL-6 expression; FIGS. 5F-5I show the inhibition of AMP12 on the mRNA expression of the inflammatory factors IL-1β, IL-6, CCL-2, and COX-2, respectively;

FIG. 6A shows the inhibition of AMP12 variants on Substance P-induced degranulation; FIG. 6B shows the inhibition of AMP12 variants on Substance P-induced IL-1β expression; FIG. 6C shows the inhibition of AMP12 variants on Substance P-induced IL-6 expression; and

FIGS. 7A-7E show the peptide AMP12 and its variants AIP1/3/4/8 can bind TLR2 protein efficiently.

### DETAILED DESCRIPTION

To further illustrate the disclosure, embodiments detailing a peptide exhibiting both antibacterial and anti-inflammatory activities, and applications thereof are described below. It should be noted that the following embodiments are intended to describe and not to limit the disclosure.

In one aspect, the disclosure provides a therapeutic peptide (isolated). As used herein, “peptide” refers to a polypeptide consisting of 18 to 25 amino acid residues in length. The peptide may be produced by any suitable method, such as chemical synthesis. Any hydrogen atom in a peptide of the disclosure may be substituted with deuterium or tritium. The peptide could exhibit a net positive charge (i.e., cationic) in aqueous solution at pH 7.0.

In some embodiments, the peptide comprises SEQ ID NO: 1, or a variant (AIP1/3/4/8) thereof having ≥88% sequence identity with SEQ ID NO: 1, and optionally ≥93% sequence identity therewith.

AMP12:
(SEQ ID NO: 1)
KKWRKVLKLIKRLVG.
AIP1:
(SEQ ID NO: 2)
KKWRKVLKFIKRLV.
AIP3:
(SEQ ID NO: 3)
KKWRKVLKFIKRL.
AIP4:
(SEQ ID NO: 4)
KKWRKVGKLIKRLVG.
AIP8:
(SEQ ID NO: 5)
KKWRKVLDLIKRLV.

The peptide of the disclosure may exhibit antimicrobial activity against one or more species of microbes (e.g., bacteria, fungi, parasites, etc.). The peptide may be effective against one or more Gram-positive bacteria, one or more Gram-negative bacteria, or both. Additionally, the peptide may be effective against both bacteria and fungi. Examples of bacteria against which the peptide may be effective include Candida albicans, Propionibacterium acnes, Escherichia coli, and Staphylococcus aureus.

The peptide of the disclosure may also possess anti-inflammatory activity. The peptide may reduce blood biomarkers of inflammation, such as C-reactive protein (CRP), lipid peroxidation products (e.g., prostaglandins), fibrinogen, and pro-inflammatory cytokines (e.g., IL-1, IL-6, TNF-α, etc.). Anti-inflammatory action may be indicated by blood biomarkers, such as a decrease in nitric oxide levels and an increase in anti-inflammatory cytokines (e.g., IL-10, IL-4, TGF-β, IL-1 receptor antagonist, etc.).

For therapeutic purposes, the peptide of the disclosure may be provided in any suitable form for delivery via any appropriate route of administration, such as injection, oral, topical, etc. In some embodiments, the peptide is provided in an aqueous solution that can be delivered by injection (e.g., intravenous or intramuscular). The concentration of the peptide in the aqueous solution may range from 3 to 30 mg/mL. The aqueous solution (containing the peptide) may be provided in a medication container as a single-use product. This medication container may be any suitable type of container for holding medications, such as a vial, ampoule, bottle, pre-filled syringe, bag, etc. The volume of the aqueous solution (containing the peptide) in the medication container may be ≤30 mL, and optionally ≥1.0 mL.

In some embodiments, the peptide is provided in solid powder form (e.g., as a lyophilized powder) within a medication container as a single-use product. For use, the peptide (in powder form) is reconstituted into an aqueous solution and delivered via injection into the patient. In this context, the amount of peptide in the medication container may range from 200 to 1,200 mg.

In some embodiments, the peptide is provided in a topical composition. Examples include ointments, gels, creams, lotions, foams, pastes (e.g., toothpaste), skin patches, sprays, and aqueous solutions (e.g., mouthwash). In this context, the concentration of the peptide in the topical composition may range from 0.5 to 25 ppm (parts per million), and optionally from 2.0 to 10 ppm.

For convenient use, the topical composition may be provided in a topical medication dispenser (such as a tube or bottle). The medication dispenser may contain 20 to 250 grams of the topical composition, expressed in weight. For example, the product could be a dispensing tube containing 60 grams of the topical composition. In the context of a topical aqueous solution, the medication dispenser may contain 15 to 1,500 mL of the topical composition. For instance, the product could be a mouthwash solution with 1.0 liter contained in a bottle.

In another aspect, the disclosure relates to a method for treating a medical condition in a patient. Any relevant medical condition may be treated. For example, the medical condition could be acne. In this context, the peptide may be effective in treating (by topical application) both the bacterial infection (e.g., Propionibacterium acnes, Candida albicans, etc.) and the resulting inflammation. Other medical conditions that may be treated include additional pathogenic bacterial or fungal infections.

The method comprises administering a therapeutically effective amount of a peptide of the disclosure to the patient. The peptide may be administered to the patient by injection (e.g., intravenous or intramuscular). In some embodiments, the amount of peptide administered to the patient (as a single dose) ranges from 2 to 60 mg/kg of body weight. In other embodiments, the amount of peptide administered to the patient (as a single dose) ranges from 200 to 2,500 mg.

The peptide may also be administered topically, such as on the skin, mucosa, or in a body cavity (e.g., oral, nasal, anal, vaginal, etc.). In this context, the method may comprise applying 2.0 to 50 grams of the topical composition to the treatment area.

### Example 1

The Inhibitory Effect of AMP12 on Propionibacterium acnes (ATCC: 6919)

Experimental Steps:

(1) Propionibacterium acnes was inoculated into Wilkins Chalgren liquid medium and cultured overnight under anaerobic conditions at 37° C. (Labiophy HL-B fully automated anaerobic workstation).

(2) 200 μL of the Wilkins Chalgren liquid culture medium and a predetermined concentration of the peptide AMP12 were added to a 96-deep-well plate. Then, 10 μL of Propionibacterium acnes was inoculated (mixed well before inoculation).

(3) After 48 hours of cultivation under anaerobic conditions at 37° C., the turbidity of the bacterial solution was observed. Then, 100 μL of the solution was taken, and the OD at 630 nm was measured using the enzyme-linked immunosorbent assay (ELISA) reading plate (after multiple blows and mixing).

$$Calculate the Percentage of Bacterial Viability = ( OD measurement - ODneg ) / ( ODpos - ODneg ) * 100 \qquad ( 4 )$$

OD Measurement: OD value of the test peptide.

ODneg: OD value with no bacterial inoculation, containing only LB medium.

ODpos: OD value with bacterial inoculation and without inhibitors.

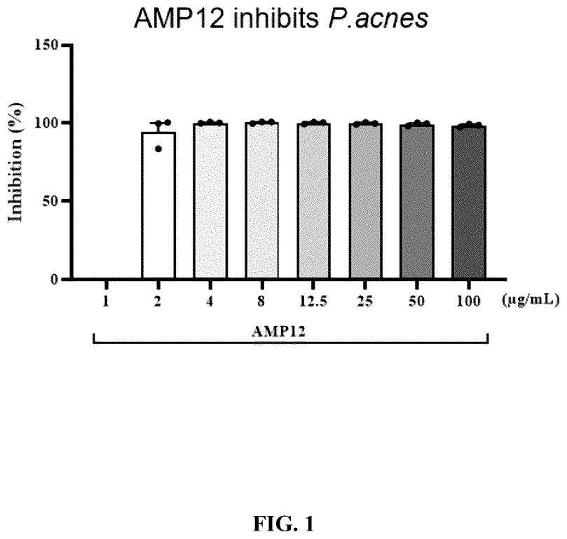

As shown in FIG. 1, at concentrations exceeding 2 μg/mL, AMP12 inhibits Propionibacterium acnes by over 90%.

### Example 2

The Inhibitory Effect of AMP12 on Escherichia coli (ATCC: 25922) and Staphylococcus aureus (ATCC: 25913)

(1) Staphylococcus aureus and Escherichia coli were inoculated into LB liquid medium and cultured overnight under aerobic conditions at 37° C. and 1800 rpm on a shaker.

(2) 200 μL of LB liquid culture medium and a predetermined concentration of peptide AMP12 were added to a 96-deep-well plate. Then, 10 μL of the Staphylococcus aureus/Escherichia coli solution was inoculated (mixed well before inoculation).

(3) After incubating at 1800 rpm for 24 hours on a shaking table at 37° C., the turbidity of the bacterial solution was observed. A volume of 100 μL of the solution was taken, and the OD at 630 nm was measured using an enzyme-linked immunosorbent assay (ELISA) reader (after multiple blows and mixing).

$$Calculate the Percentage of Bacterial Viability = ( OD measurement - ODneg ) / ( ODpos - ODneg ) * 100. \qquad ( 4 )$$

OD Measurement: OD value of the test peptide.

ODneg: OD value with no bacterial inoculation, containing only LB medium.

ODpos: OD value with bacterial inoculation and without inhibitors.

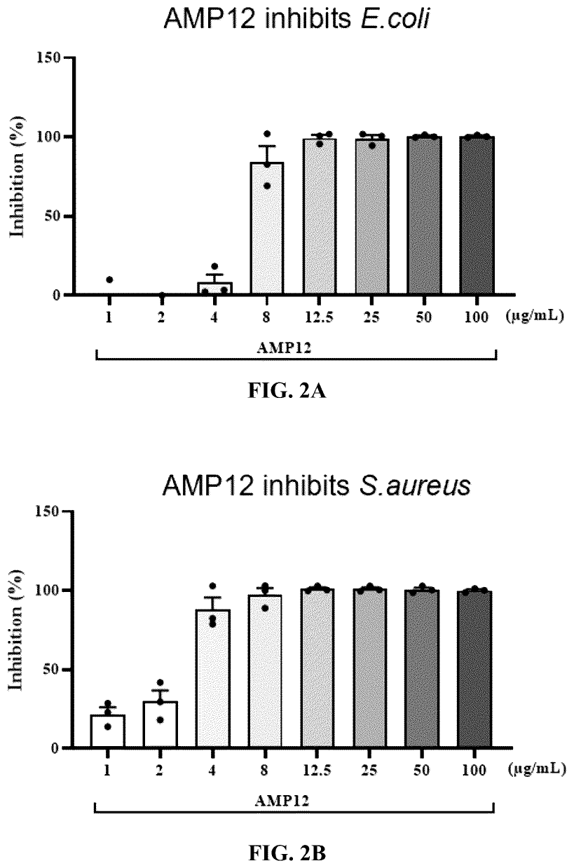

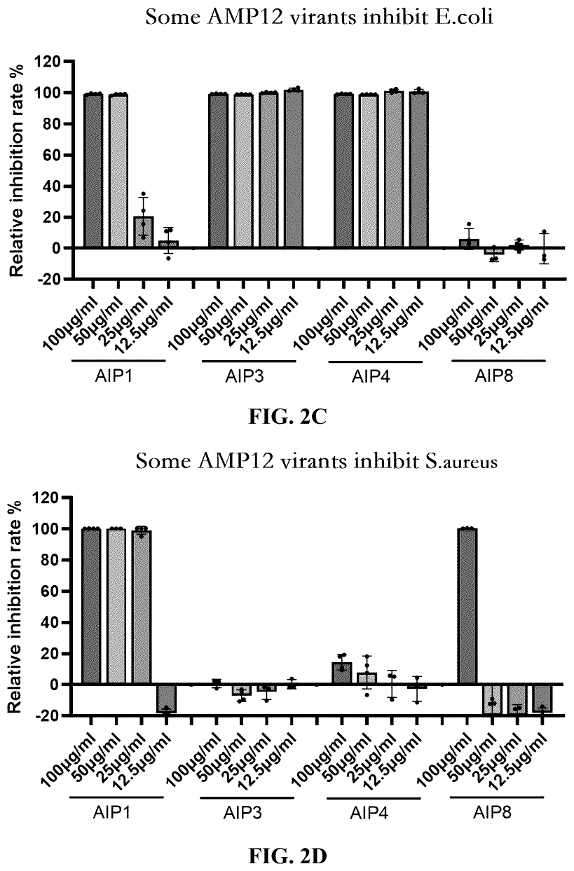

As shown in FIG. 2A, AMP12 completely (100%) inhibits Escherichia coli at concentrations exceeding 12.5 μg/Ml; as shown in FIG. 2B, AMP12 completely inhibits Staphylococcus aureus at concentrations exceeding 8 μg/mL.

As shown in FIG. 2C, the inhibitory effect of the AMP12 variants on Escherichia coli. Specifically, the variant AIP1 completely inhibits Escherichia coli at concentrations exceeding 50 μg/mL, and the variants AIP3 and AIP4 completely inhibits Escherichia coli at concentrations exceeding 12.5 μg/mL.

As shown in FIG. 2D, the inhibitory effect of the AMP12 variants on Staphylococcus aureus. Specifically, the variant AIP1 completely inhibits Staphylococcus aureus at concentrations exceeding 25 μg/mL, and the variant AIP8 completely inhibits Staphylococcus aureus at concentrations exceeding 100 μg/mL.

### Example 3

The Inhibitory Effect of AMP12 on Candida albicans (ATCC: 14053)

(1) Candida albicans was inoculated into YM liquid medium and cultured overnight under aerobic conditions at 28° C. and 1800 rpm on a shaker.

(2) 200 μL of YM liquid culture medium and a predetermined concentration of peptide AMP12 were added to a 96-deep-well plate. Then, 10 μL of the Candida albicans solution was inoculated (mixed well before inoculation).

(3) After incubating at 1800 rpm for 28 hours on a shaking table at 28° C., the turbidity of the bacterial solution was observed. A volume of 100 μL of the solution was taken, and the OD at 630 nm was measured using an enzyme-linked immunosorbent assay (ELISA) reader (after multiple blows and mixing).

$$Calculate the Percentage of Bacterial Viability = ( OD measurement - ODneg ) / ( ODpos - ODneg ) * 100 \qquad ( 4 )$$

OD Measurement: OD value of the test peptide.

ODneg: OD value with no bacterial inoculation, containing only LB medium.

ODpos: OD value with bacterial inoculation and without inhibitors.

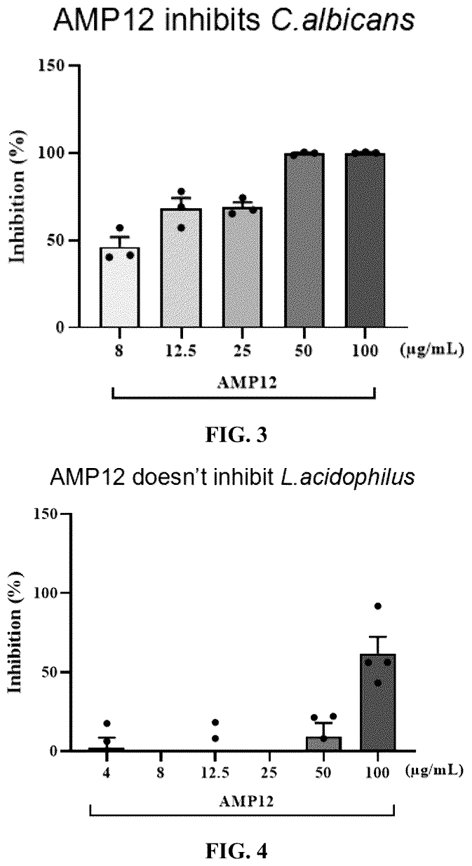

As shown in FIG. 3, AMP12 completely (100%) inhibits Candida albicans at concentrations exceeding 12.5 μg/mL.

### Example 4

Inhibition of AMP12 on Lactobacillus acidophilus (ATCC: 4356)

Experimental Steps:

(1) Lactobacillus acidophilus was inoculated into MRS liquid medium and cultured overnight under anaerobic conditions at 37° C. (Labiophy HL-B fully automated anaerobic workstation).

(2) 100 μL of MRS liquid culture medium and a predetermined concentration of peptide were added to a 96-deep-well plate. Then, 100 μL of Lactobacillus acidophilus was inoculated (mixed well before inoculation).

(3) After 48 hours of cultivation under anaerobic conditions at 37° C., the turbidity of the bacterial solution was observed. A volume of 100 μL of the solution was taken, and the OD at 630 nm was measured using an enzyme-linked immunosorbent assay (ELISA) reading plate (after multiple blows and mixing).

$$Calculate the Percentage of Bacterial Viability = ( OD measurement - ODneg ) / ( ODpos - ODneg ) * 100 \qquad ( 4 )$$

OD Measurement: OD value of the test peptide.

ODneg: OD value with no bacterial inoculation, containing only LB medium.

ODpos: OD value with bacterial inoculation and without inhibitors.

The results are shown in FIG. 4, indicating that AMP12 does not inhibit the growth of Lactobacillus acidophilus.

### Example 5

### Anti-Inflammatory Effect of AMP12 on Mouse Macrophage RAW264.7 Cells

Experimental Steps:

(1) Mouse macrophage RAW264.7 cells were cultured in DMEM supplemented with 10% fetal bovine serum (FBS) and 1% penicillin and streptomycin (P/S) at 37° C. in a 5% CO₂ incubator.

(2) 5×10⁴ mouse-derived RAW264.7 macrophages were placed in 12-well cell culture plates and cultured for 24 hours. Then, 20 μL of PBS (negative control), Dexamethasone (positive control), and the test peptide were added to the wells for incubation for 2 hours. Afterward, 10 ng/ml of lipopolysaccharide (LPS) was mixed with RAW264.7 cells and incubated for 24 hours.

(3) The supernatant was collected, and NO and IL-6/IL-1β expression levels were measured using a commercial kit.

(4) The cells were collected, and total RNA was isolated and extracted, reverse transcribed, and subjected to RT-qPCR to detect inflammation markers, including TNF-α, IL-1β, IL-6/10, CCL2, IFN-γ, iNOS, and GAPDH.

Experimental results:

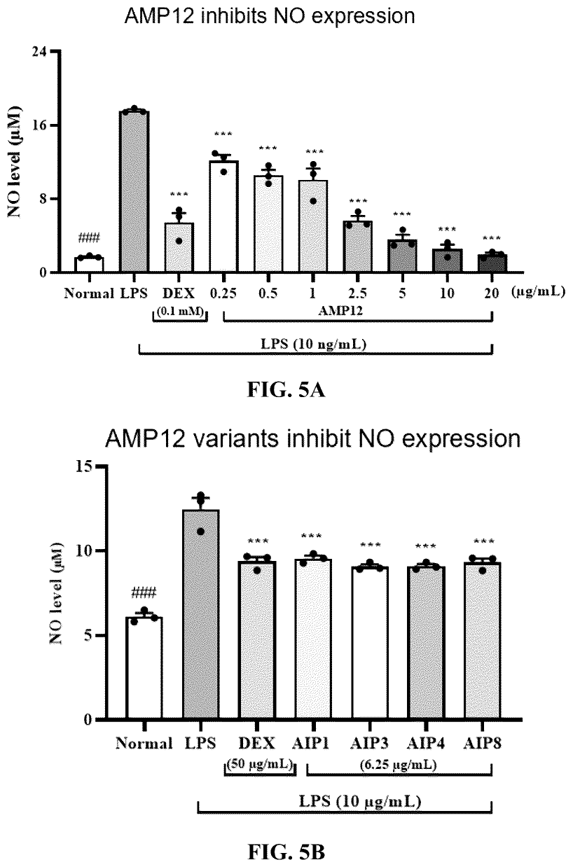

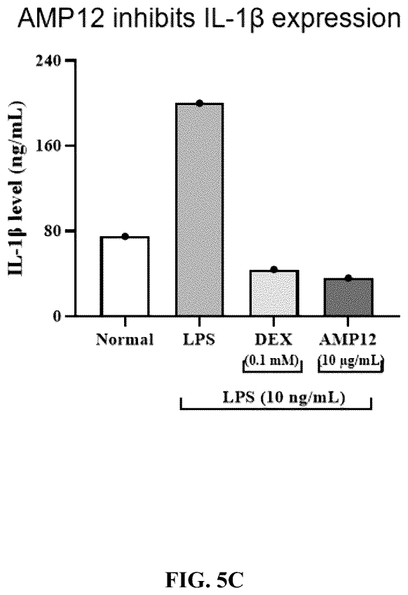

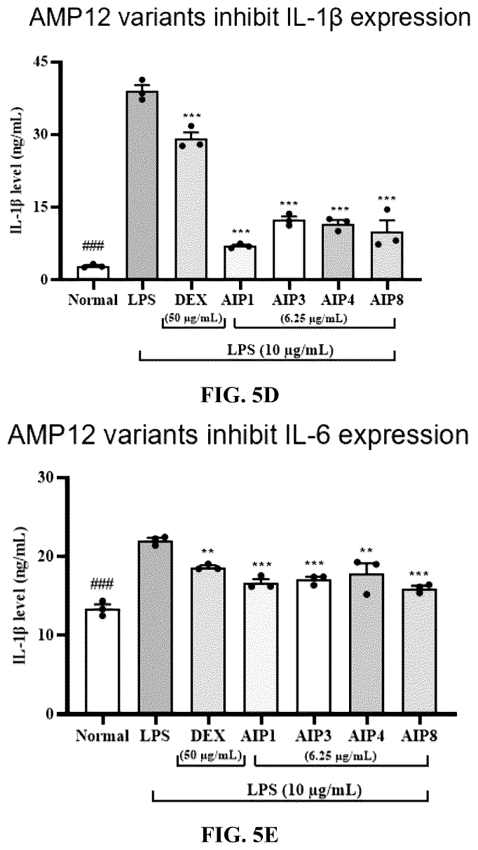

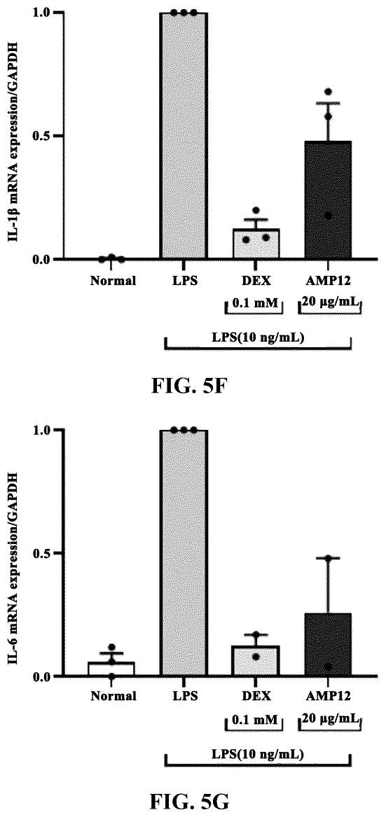

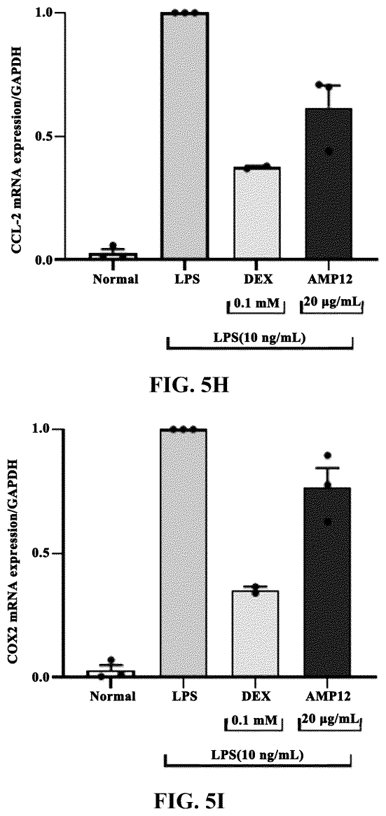

1. After treating RAW264.7 cells with the peptide AMP12 and its variants AIP1/3/4/8, as shown in FIGS. 5A-5B, AMP12 effectively inhibited NO concentration even at a low concentration (0.25 μg/mL); the variants AIP1/3/4/8 effectively inhibited NO concentration at low concentrations (6.25 μg/mL).

2. After treating RAW264.7 cells with the peptide AMP12 and its variants AIP1/3/4/8, an ELISA kit was used to detect the concentration of IL-6 and IL-1β proteins. As shown in FIGS. 5C-5E, AMP12 inhibited the expression of IL-1β at a low concentration (10 μg/mL); the variants AIP1/3/4/8 inhibited the expression of IL-6 and IL-1β proteins at low concentrations (6.25 μg/mL).

3. After treating RAW264.7 cells with AMP12, RT-qPCR was used to detect the mRNA levels of inflammatory factors. As shown in FIGS. 5F-5I, AMP12 inhibited the mRNA expression of the inflammatory factors IL-1β, IL-6, CCL-2, and COX-2 at a concentration of 20 μg/mL.

### Example 6

### Immunomodulatory Effect of Peptide AMP12 Variant on P815 Cells

Experimental steps:

1) Cell Culture of P815 Mast Cell Tumor in Mice: P815 cells were cultured in DMEM high-glucose medium (containing 10% fetal bovine serum, 2 mM glutamine, and 1% penicillin/streptomycin) at 37° C. in a 5% CO₂ incubator, and passaged every 2-3 days. The logarithmic growth phase P815 cells were collected for subsequent experiments. The P815 cells were plated in a 96-well plate at a density of 10,000 cells per well. After the cells were adhered for about 1 hour, an inhibitor (AMP12/sodium tryptophan SC) was added, followed by the addition of the inducer SP (0.1 μM) for 24 hours.

2) The solution was centrifuged, and the supernatant was collected for further tests.

3) The operation for determining the content of β-hexosaminidase (ELISA, Keaibo Biotechnology Co., Ltd.), IL-1β, IL-6 (ELISA, Guandong Biotechnology Co., Ltd.) was carried out according to the instructions of the reagent kit.

4) 0.1% Triton X-100 was added to the blank control group to induce cell lysis for 5 minutes, and the resulting solution was centrifuged to obtain a cell lysate.

Total β-hexosaminidase level was calculated as follows: A₄₅₀ (Cell supernatant of the blank control group)+A₄₅₀ (Cell lysate of the blank control group), Therefore, for the blank control group, both the supernatant and the underlying cell lysate must be collected and assayed separately for β-hexosaminidase content.

β-hexosaminidase level for other treatment groups: A₄₅₀ (Cell supernatant of the respective treatment group).

$$β - hexosaminidase release rate = ( {A}_{450} ( Cell supernatant of the respective treatment group ) / ( {A}_{450} ( Cell supernatant of the blank control group ) + ( {A}_{450} ( Cell lysate of the blank control group ) ) \times 100 \% .$$

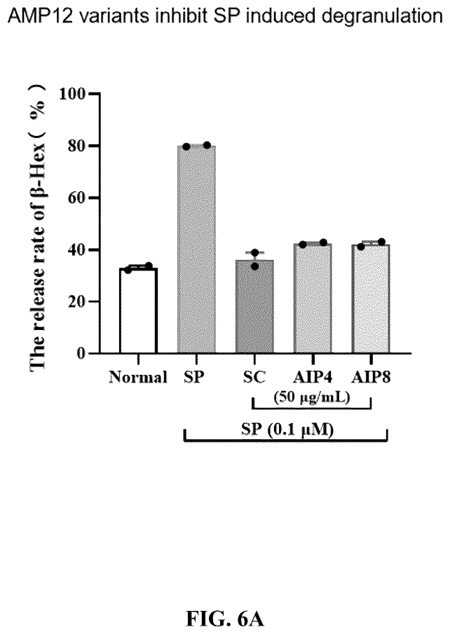

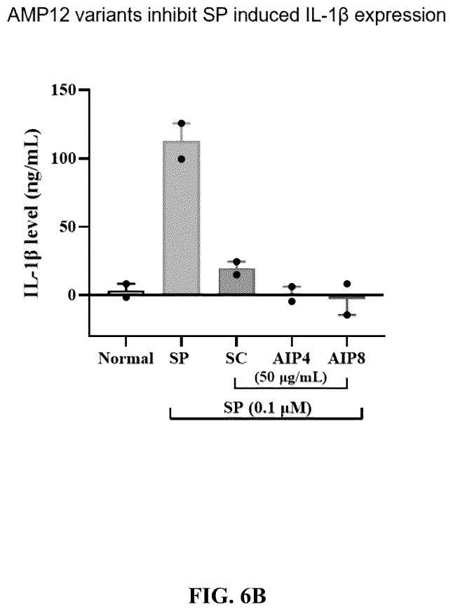

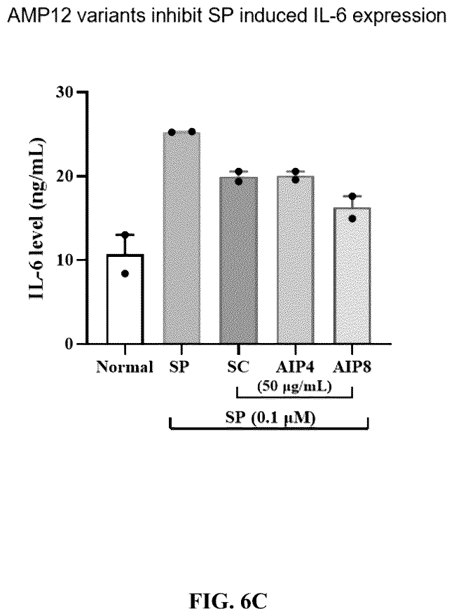

As shown in FIGS. 6A-6C, at a concentration of 50 μg/mL, AMP12 reduced Substance P-induced inflammatory allergic responses, and decreased the release of β-hexosaminidase and the levels of IL-6 and IL-1β.

### Example 7

### Binding Affinity of AMP12 Variants to Toll-Like Receptor 2 (TLR2)

The binding affinity between the peptide and Toll-like Receptor 2 (TLR-2) was assessed via Surface Plasmon Resonance (SPR) using a Biacore T200 instrument (GE Healthcare) and a CM5 sensor chip (GE Healthcare) at ambient temperature. TLR-2, diluted to 0.02 mg/mL in sodium acetate buffer (pH 4.5; GE Healthcare), was immobilized onto the CM5 chip. The chip surface was activated with a 1:1 volume ratio of N-hydroxysuccinimide (NHS) and 1-ethyl-3-(3-dimethylaminopropyl)carbodiimide (EDC), and then blocked with ethanolamine. The ligand adsorbed to the chip surface was regenerated using 50 mM sodium hydroxide. The peptides diluted in PBS-P buffer (GE Healthcare) to concentrations ranging from 62.5 nM to 2000 nM were flowed over the chip surface at a rate of 30 μL/min for an association phase of 120 seconds, followed by a dissociation phase of 120 seconds using running buffer PBS-P. The binding affinity was determined using Biacore Evaluation Software (GE Healthcare).

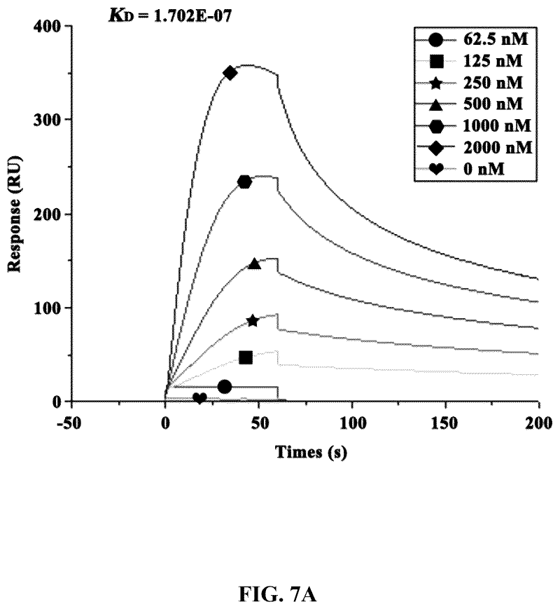

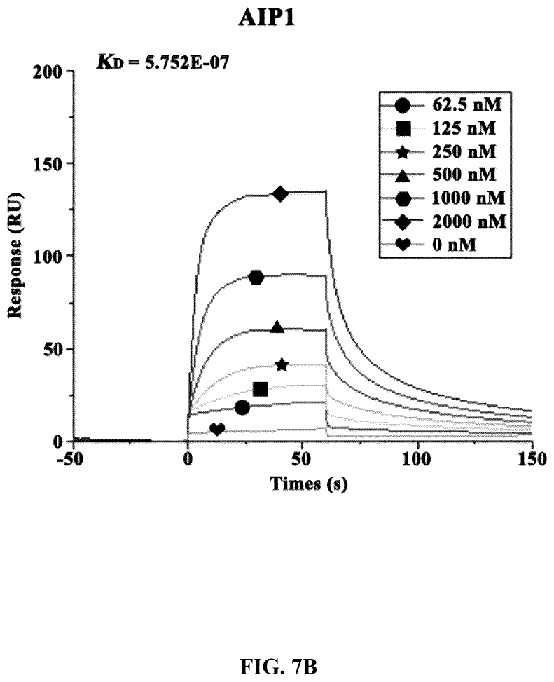

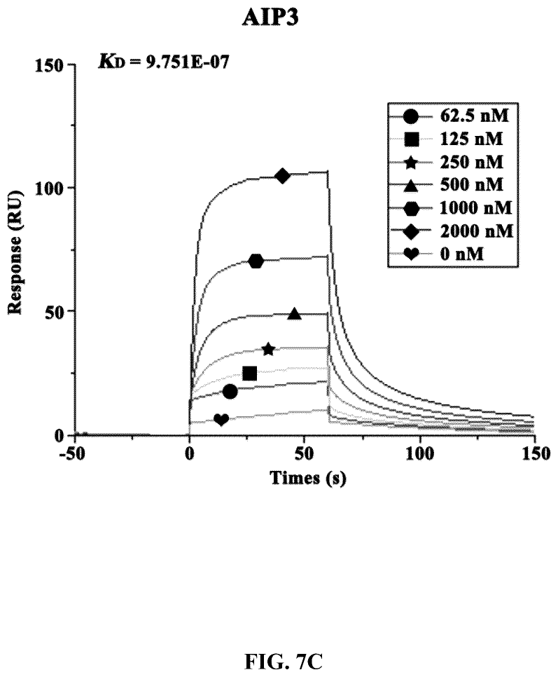

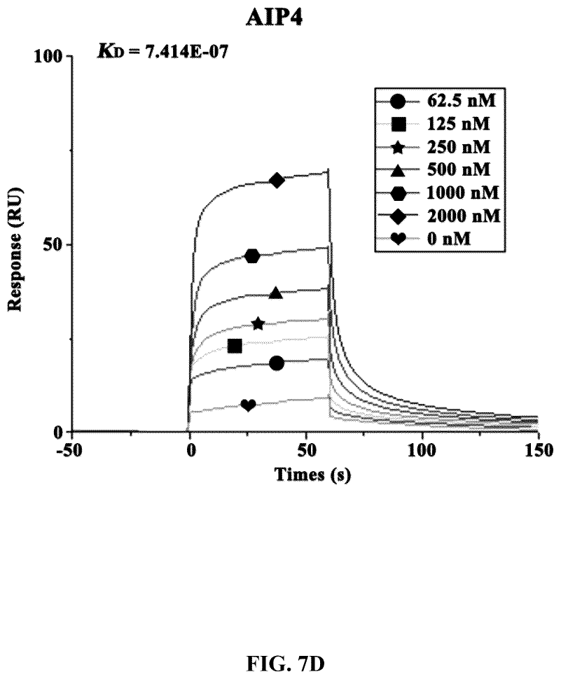

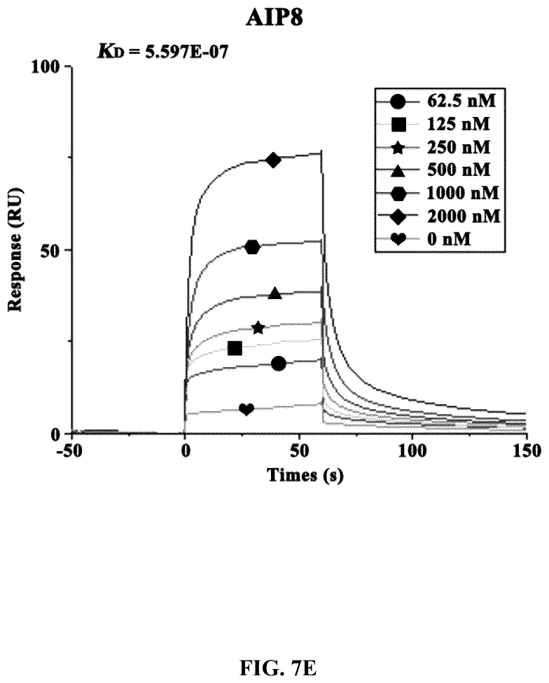

As shown in FIGS. 7A-7E and Table 1, the peptide AMP12 and its variants AIP1/3/4/8 all exhibited low micromolar affinity for the TLR2 protein.

TABLE 1
Binding affinity of AMP12 and its variants to TLR2

| ID | Sequence | K_{D} (UM) |
|---|---|---|
| AMP12 | KKWRKVLKLIKRLVG (SEQ ID NO: 1) | 0.1702 |
| AIP1 | KKWRKVLKFIKRLV (SEQ ID NO: 2) | 0.3759 |
| AIP3 | KKWRKVLKFIKRL (SEQ ID NO: 3) | 0.6311 |
| AIP4 | KKWRKVGKLIKRLVG (SEQ ID NO: 4) | 0.3841 |
| AIP8 | KKWRKVLDLIKRLV (SEQ ID NO: 5) | 0.5597 |

The dissociation constant (K_{D}) is a crucial indicator in SPR experiments, representing the degree of dissociation between a ligand and a protein receptor at equilibrium, which is inversely proportional to affinity. A higher K_{D} indicates a greater degree of dissociation, meaning weaker affinity between the ligand and the protein receptor. Typically, a K_{D} value within the range of 10⁻⁶ to 10⁻⁹ mol/L is considered to indicate a strong affinity between a protein and a small molecule.

Conclusion: The peptide AMP12 and its variants exhibit potent inhibitory activity against pathogenic microorganisms, but show no significant inhibition against the probiotic Lactobacillus acidophilus. Furthermore, they significantly suppress the expression of inflammatory responses in LPS-induced RAW264.7 cells and inhibit the sensitization effect of Substance P on P815 mast cells. These peptides exhibit strong binding affinity for TLR2, suggesting that their aforementioned effects are likely mediated through TLR2.

It will be obvious to those skilled in the art that changes and modifications may be made, and therefore, the aim in the appended claims is to cover all such changes and modifications.

What is claimed is:

1. A peptide or a derivative thereof, comprising an amino acid sequence of SEQ ID NO: 1, or a variant thereof.

2. The peptide or a derivative thereof of claim 1, wherein the variant of SEQ ID NO: 1 has ≥88% sequence identity with SEQ ID NO: 1.

3. The peptide or a derivative thereof of claim 1, wherein the amino acid sequence of SEQ ID NO: 1 comprises an N-terminus; a hydrogen atom of the N-terminus is substituted by CH₃CH(OH)CO— or R₁—CO—, and R₁ is selected from a group consisting of hydrogen, hydroxyl, amino, an alkyl group, and an alkenyl group.

4. The peptide or a derivative thereof of claim 1, wherein the amino acid sequence of SEQ ID NO: 1 comprises a C-terminus; a hydrogen atom of the C-terminus is substituted by —NR₂R₃ or —OR₂, and R₂ and R₃ are independently selected from a group consisting of hydrogen, hydroxyl, amino, an alkyl group, and an alkenyl group.

5. The peptide or a derivative thereof of claim 3, wherein the alkyl group is selected from a group consisting of methyl, ethyl, isopropyl, isobutyl, tert-butyl, pentyl, hexyl, heptyl, octyl, decyl, 2-ethylhexyl, 2-methylbutyl, and 5-methylhexyl.

6. The peptide or a derivative thereof of claim 4, wherein the alkyl group is selected from a group consisting of methyl, ethyl, isopropyl, isobutyl, tert-butyl, pentyl, hexyl, heptyl, octyl, decyl, 2-ethylhexyl, 2-methylbutyl, and 5-methylhexyl.

7. The peptide or a derivative thereof of claim 3, wherein the alkenyl group is selected from a group consisting of vinyl, linoleyl, and oleyl.

8. The peptide or a derivative thereof of claim 4, wherein the alkenyl group is selected from a group consisting of vinyl, linoleyl, and oleyl.

9. A pharmaceutical composition, comprising:
a medication container; and
a therapeutically effective amount of the peptide or a derivative thereof of claim 1.

10. The pharmaceutical composition of claim 9, wherein the peptide or a derivative thereof is in solid powder form in an amount of 200-2,500 mg per dose.

11. The pharmaceutical composition of claim 9, further comprising an aqueous solution contained in the medication container; wherein the peptide or a derivative thereof is dissolved in the aqueous solution at a concentration of 3-30 mg/mL.

12. A method for treating a medical condition in a patient, comprising administering to the patient a therapeutically effective amount of the peptide or a derivative thereof of claim 1.

13. The method of claim 12, wherein an amount of the peptide or a derivative thereof is administered as a single dose is 200-2,500 mg or 2-60 mg/kg of body weight.

14. The method of claim 12, wherein the medical condition is acne and the peptide or a derivative thereof is administered topically to a treatment area on the patient's skin.

15. A method for preparing a cosmetically or pharmaceutically acceptable salt comprising applying a peptide or a derivative thereof of claim 1.

16. The method of claim 15, wherein the salt is formed by reacting the peptide or a derivative thereof of claim 1 with an organic base, and the organic base is ethylamine, diethylamine, arginine, lysine, histidine or piperazine.

17. The method of claim 15, wherein the salt is formed by reacting the peptide or a derivative thereof of claim 1 with an inorganic acid or an organic acid; the organic acid is acetic acid, citric acid, malonic acid, maleic acid, tartaric acid, fumaric acid, benzoic acid, succinic acid, oxalic acid, or gluconic acid; and the inorganic acid is hydrochloric acid, sulphuric acid, boric acid, or carbonic acid.
